(19) United States
(12) Patent Application Publication
(10) Pub. No.: US 2004/0214260 A1
(43) Pub. Date: Oct. 28, 2004

(54) Mold test Kit

(76) Inventor: Craig Spiesman, Monroe, NC (US)

Correspondence Address: DOUGHERTY, CLEMENTS & HOFER, 1901 ROXBOROUGH ROAD, SUITE300, CHARLOTTE, NC, 28211, US

(21) Appl. No.: 10/828,567

(22) Filed: Apr. 21, 2004

Related U.S. Application Data
(60) Provisional application No. 60/465,333, filed on Apr. 25, 2003.

(51) Int. Cl. C12Q 1/04; C12M 1/34

(52) U.S. Cl. 435/34; 435/287.1

(57) ABSTRACT

A mold test kit comprising: a) a foldable sheet of paperboard having leafs that can be folded into a multi-ply folder/mailer; b) at least one paperboard slide perforatedly attached to the paperboard, said slide having a window with a pane that is a relatively tacky adhesive supported on a clear film; c) a removable cover slip that covers the window; d) a reversibly attachable slide mounting zone with a filmic release layer for sending slides having sampled mold to a laboratory for analysis; and e) printed instructions for use.

### CROSS REFERENCE TO RELATED APPLICATION

The application claims the benefit of the priority filing date of the U.S. provisional patent application filed on Apr. 25, 2003, bearing Ser. No. 60/465,333.

### BACKGROUND OF THE INVENTION

1) Field of the Invention

The invention relates to a mold test kit and more particularly to a mailable mold test kit that is suitable for use by non-professionals. The kit contains all the components necessary to collect mold samples, and a means for transporting the samples to a lab for analysis.

2) Prior Art

Heretofore, one method for collecting mold samples used a roll of single sided transparent pressure sensitive tape (PSA), such as scotch tape or the like. The user would press a non-standardized section of the tape on the unidentified mold (e.g., the mold to be tested), wherein the mold would adhere to the PSA tape. Typically, the tape is then adhered to a glass slide, carefully packaged, and sent to the laboratory for analysis. Several problems exist with this method. Special packaging must be employed to prevent the glass slides (and even some plastic slides) from being damaged during transportation. Also, there is no easy method that the slide can be identified with the sampling record which information on when, where and how the sample was collected. Another problem is that during laboratory analysis, when the tape is peeled off the slide to expose the mold on the adhesive slide, the tape infrequently tears during removal. In an even worse scenario, the tape does not have sufficient adhesion and it detaches from the slide during transportation, therein dislodging the mold from the adhesive, and possibly cross-contaminating other test adhesive strips packaged with it.

What is desired is a mold test kit that has a tape with a standardized sample area, a non-friable slide that does not require special packaging during shipping, a kit having several mold collection test strips that are uniquely identified with a control number that references a record of when, where, and by whom the sample was collected, and a kit that utilizes a process of collecting the mold samples that can be performed by a non-professional. Also, what is needed is a kit where the samples can be expeditiously analyzed by the laboratory. Important criteria include a kit where the sample can be prepared for viewing under a microscope without the associated problems previously identified with removing the tape from the slide, a sample that is easily associated with the record identifying the history of the sample, and a sample that can be stored for reevaluation/further analysis, if required in the future. The kit preferably is a compact protective means for storing the samples that maintains the integrity of the sample and the record.

### SUMMARY OF THE INVENTION

The mold test kit is comprised of at least one paper slide having a clear filmic window, wherein the window defines a perimeter of a coating of a relatively high tack adhesive. The paper slide is printed with identifying indicia, which uniquely correlate the slide to a kit and to a sample log that designates pertinent information as to the collection of the sample. The high tack adhesive, prior to collecting the mold sample, is covered with a cover slip that is a releasable filmic strip that is preferably clear. The paper slide is comprised of a relatively heavy weight stock, single-ply, printable paperboard. The slides are preferably attached to a creased paperboard folder/mailer that simultaneously serves as a protective multi-fold sleeve for mailing new slides to a user, a sample log for recording the sampling process, and a source of printed instructions on how to collect mold samples and symptoms associated with various molds. The paperboard folder/mailer also is a means for mailing the sample slides to the laboratory for analysis. The folder/mailer is formed by inwardly folding the paperboard sheet, which is creased forming four leafs, thereby enclosing the slides on the interior of a relatively stiff multi-ply folder/mailer. Slides are preferably attached to an edge of the paperboard folder/mailer and are easily, singularly, detached along a perforated edge. To mail the folder/mailer to the laboratory for analysis, the kit has a slide mount adhesive on an interior side of the paperboard for reversibly attaching the paper slide(s). The slide mount adhesive is initially covered with a release liner, wherein the release liner can be partially pulled away, therein exposing the slide mount adhesive aligned along a target zone for fastening the slide with the sampled mold. The target zone preferably has a filmic material having release properties that prevent the high tack adhesive in the window from adhering to the target zone. After collecting a sample of mold, the slide is placed mold side down, where an edge of the slide is adhesively fastened to the slide mount adhesive. The slide mount adhesive is preferably a pressure sensitive adhesive that enables the slide to be reversibly attached. The window of the slide is positioned such that the window overlays the filmic material having release properties. In a typical configuration, the paperboard folder/mailer would be folded into approximately three equal width leafs, and along an edge of one of the leafs, would be a fourth leaf to which there is attached at least one slide with a perforated edge connecting the slide(s) to the folder/mailer. The folder/mailer is printed with instructions on how to take the samples, instructions on how to return the sample to the laboratory, and usually information on mold. The kit can be laid out as follows. The slide mount adhesive is substantially located on the interior side, preferably on a leaf near a center leaf. The slide mount adhesive is adjacent to target zones for attaching the slides. The target zones are scribed with lines designating where the slide is attached.

In a typical sampling operation, after receiving the kit, the user tears off a slide along the perforations and peels off the cover slide, therein exposing the window of tacky adhesive. The user positions the window over the area being sampled and presses the back side of the slide against the sample area, therein pushing the tacky adhesive against the mold. The mold adheres to the adhesive. The user then pulls back a portion of the release liner exposing the slide mount adhesive aligned along the rectangle target zone, and presses an edge of the slide down so that the slide is in contact with the adhesive. The user then records the sampling data in the log. The process is repeated until the user is satisfied that he has collected a sufficient number of samples for analysis. The user instructs the lab where he wishes the result to be sent and how he wants the results to be posted (i.e., by telephone, email, fax or post). The paperboard is folded reforming the folder/mailer, placed into an envelope and mailed to the lab. At the lab the slide is lifted off the target strip, turned over and slid underneath the microscope so that the mold is facing upright. The mold is examined and analyzed for both type and quantity. The slide is then returned to the rectangle target zone face side down with the mold against the filmic layer having the release line. The process is repeated until all the slides are examined, and then a report is compiled and sent to the appropriate party as designated by the user. The kit can be folded back up and stored for further examination at a later time.

### BRIEF DESCRIPTION OF THE DRAWINGS

FIG. 1 is a compressed planar of the mold test kit showing the inside of the kit.

FIG. 2 is a compressed planar view of the mold test kit showing the outside of the kit.

FIG. 3 is a partial planar view of the inside of the kit with three new slides.

FIG. 4 is a partial planar view of the inside of the kit, where two of the slides have been used to collect mold samples. The collected sample slides are positioned on the slide mount adhesive. The third slide remains unused.

FIG. 5 is an enlarged planar view of the slide.

FIG. 6 is an enlarged side view of the slide.

### DETAILED DESCRIPTION OF THE INVENTION

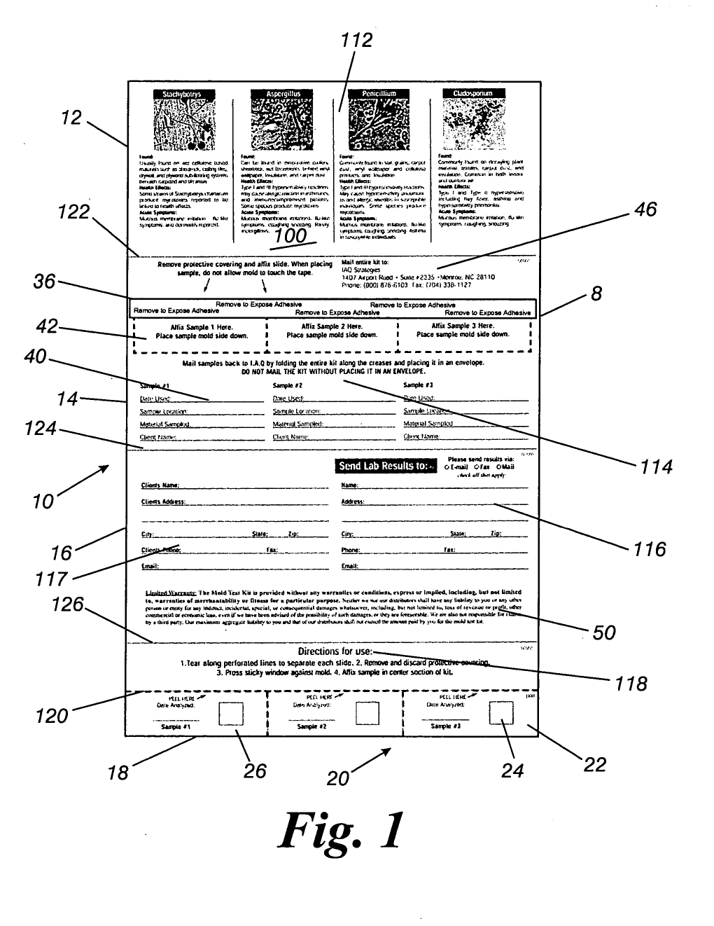

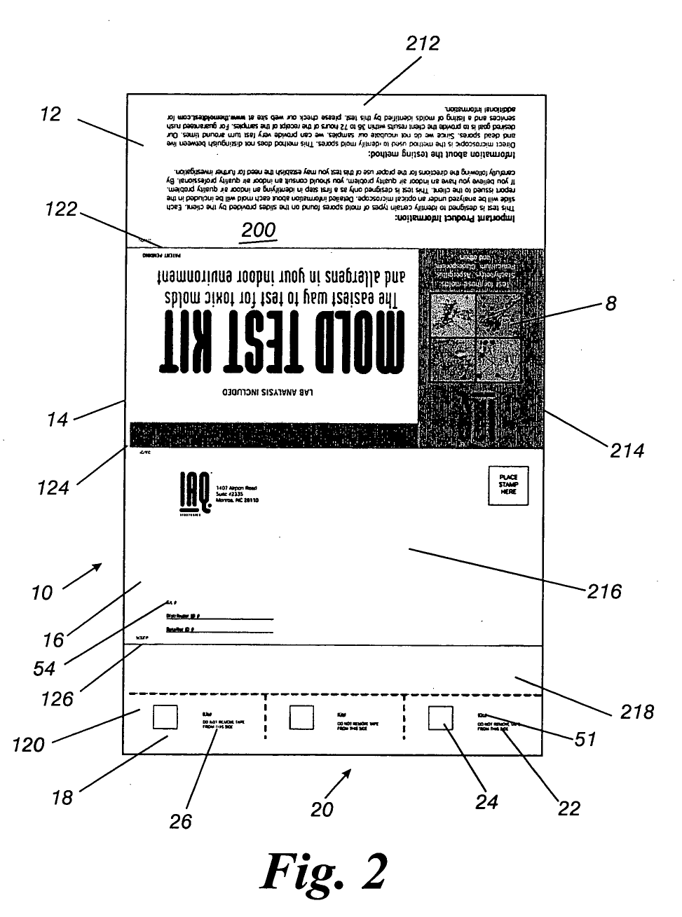

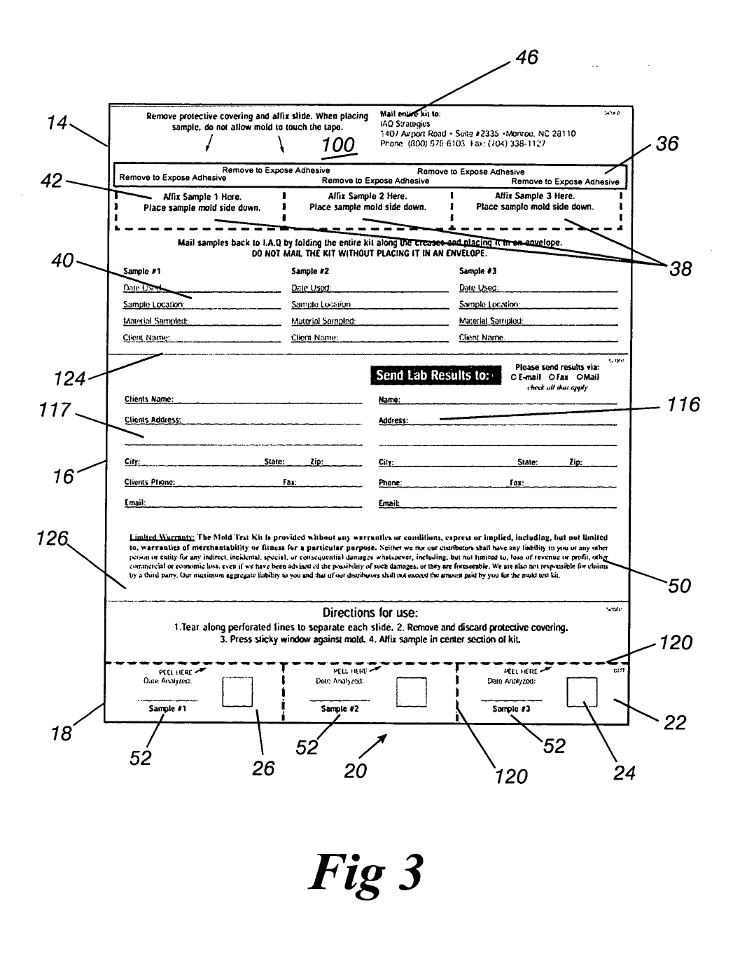

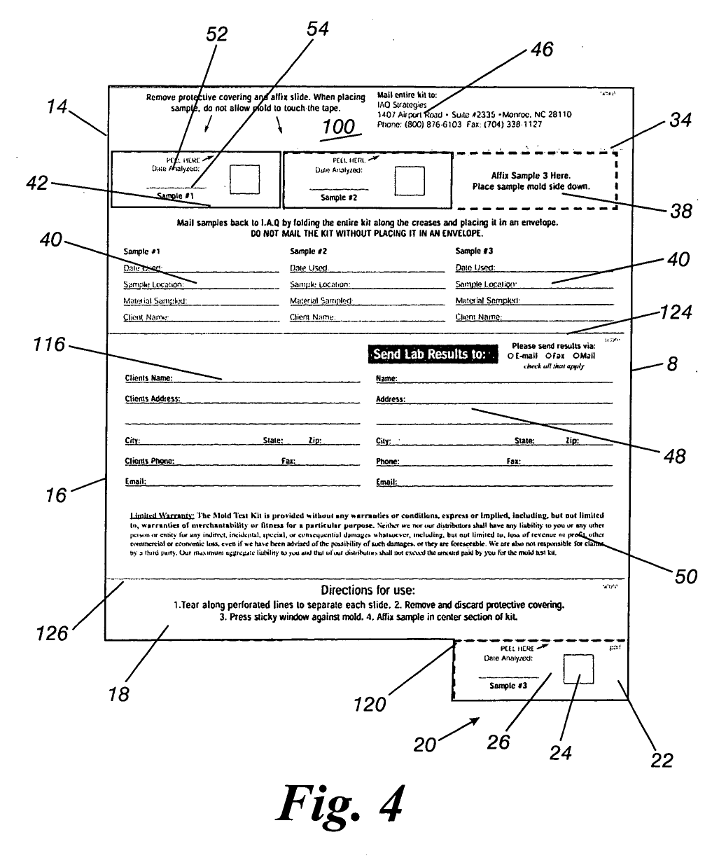

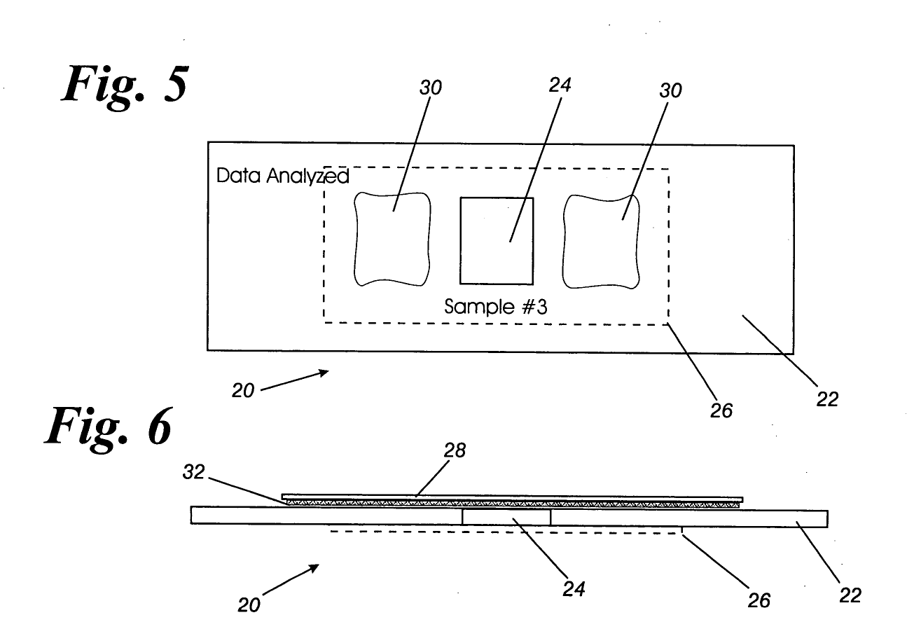

The invention is a mold test kit comprised of a single sheet of paperboard that has been printed, perforated die cut, coated, and laminated to tape; therein providing all of the elements of the test kit, as set forth herein. The single ply paperboard 8 has two sides, an inside 100 and outside 200. FIG. 1 illustrates the inside, and FIG. 2 illustrates the outside 200. Referring to FIG. 1 of test kit 10, the single sheet of paperboard 8 is creased at 122 forming a first leaf 12, and at 124 forming a second leaf 14, and at 126 forming a third leaf 16, and a fourth leaf 18. The leafs can be folded over forming a protective folder/mailer for the slides 20, which are contained within the folder/mailer for transporting and mailing the kit to the user, and to the lab for analysis of the collected samples. The leafs of the kit are roughly segregated as to content. The inside of the first leaf 12 contains information 112 on various molds, including where the molds are found, health causes, and acute symptoms associated with the mold. The second leaf 14 contains information 114 for returning the samples to the lab for analysis, and a log 40 describing when and where the samples were collected. The second leaf has rectangular target zones 42 for attaching the test slides after mold collection. The test slides 20 are mounted face down with the edge of the slide fastened to the slide mounting adhesive 34 (adhesive is covered in FIGS. 1 and 3). The slide mounting adhesive 34, which is covered with a release strip 36, is substantially a coating of adhesive running along the length of the target zones 42. The third leaf 16 contains instructions to the lab on where to send and how (i.e., email, telephone, fax) to send the lab results 116, the clients name and address 117, and warranty information 50. The fourth leaf 18 contains directions on how to detach a slide, and instructions for collecting the mold 118. In the illustrated embodiment, there are three slides 20 connected along a longitudinal edge of the fourth leaf 18. The slides 20 are fabricated as laminates of the single piece of paperboard 8, and the slides can readily be singularly separated from the fourth leaf along the perforations 120, as shown in FIGS. 3 and 4. Referring now to FIG. 2, which is a view of the outside 200 of the paperboard 8 forming kit 10, the outside of the first leaf region 212 is printed with product information, and test method information. The outside of the second leaf 214 also contains additional product information. The outside of the third leaf 216 has an address space for mailing the kit to the user, a kit ID number 54 (which also contains control numbers for distributer and retailer), and an outline for the postage stamp. The outside of the fourth leaf 218 is largely comprised of the slides 20. Each slide is printed with a kit number 51, and chain of custody 54, and a sample number 52. FIG. 3 is partial view of the inside 100 of the kit 10. The second 14, third 16, and fourth 18 leafs are shown, as well as the creases 124 and 126, which define the fold lines of the respective leafs. Along the rectangular target zones 42 for the test slides having collected mold, there is a printed single sided release liner 36. The release liner 36 is printed with the words “Remove To Expose Adhesive.” A typical release liner is a 60 pound super-calendered polyethylene coated sheet with a silicone release coat on one side. The release side goes against the slide mount adhesive 34. The slide mount adhesive 34 is selected so as to enable the slides to be detached at the lab, without tearing the paper slides 20. The slides have a mold adhesive 32 (as shown in FIG. 6) that fills window 24. The slides are loosely connected to the leaf 18 by perforated lines 120. An expanded view of a single slide is shown from a plan view in FIG. 5, and a side view in FIG. 6. The slide 20 is comprised of a heavy gauge single ply paperboard 22 that is die cut, therein forming a window 24. The pane of the window has the mold adhesive 32, which is a substantially clear high tack adhesive that is coated onto a clear filmic material 28, so as to be substantially transparent. A relatively high tack pressure sensitive tape, such as 3M S Index Scotch™ crystal clear tape, provides simultaneously both a filmic substrate 28 and a relatively high tack adhesive 32. The tape is applied to the outside of the slide 218 (FIG. 2), therein exposing the mold adhesive 32 in the window 24. The window is kept free of contamination with a cover slip 26, which is shown as dashed line in FIG. 5 and in FIG. 6. The cover slip 26 is held in placed by two relatively low tack adhesives 30, which are thin layers of adhesive deposited on either side of the window.

The method for using the kit 10 is as follows. The kit 10 is shipped to the user/client as a folder/mailer, wherein the leafs 1 and 4 are folded inwardly, and then leafs 2 and 3 are folded together and the edge is tabbed with an adhesive seal. The folded paperboard leafs create a four ply paperboard folder/mailer that is relatively stiff and protective of the slides contained therein. The user opens the kit by breaking the seal, and unfolds the single ply of paperboard until it is substantial a planar. A slide 20 is torn off along the perforations individually, just prior to sampling the site harboring the mold. In the case as shown in FIG. 4, the closet is being sampled. To collect the sample, the cover slip 26 is removed from the slide 20, therein exposing the high tack adhesive 32. The slide is aligned over the sample location, and the window is pressed until the adhesive comes in contact with the mold. The release liner 36 is pulled back exposing the slide mount adhesive 34, as shown in FIG. 4. The target zone 42 for the test slide has a filmic release layer 38 (not visible to naked eye) that spans most of the area inside the rectangle define by the rectangular target zone 42. The mold side of the window is aligned so that it overlays the filmic release layer 38, and the slide is pressed into place such that a longitudinal edge of the slide comes in contact with the slide mount adhesive 34. The user records pertinent sampling information in the spaces designated as the sample log 40. The process is repeated for each of the other two slides. The user/client then fills in the clients name and address 117, and the space 116 designating where the lab results are to be sent. The leafs of the kit are folded back and placed into a number 10 envelope and mailed to the laboratory for analysis. When the kit 10 is received by the laboratory the appropriate data, including the kit number, sample number, the user, and any intermittent distributer of the kit is recorded. The slide is reversibly removed from slide mount adhesive and placed on a microscope mold side up. The transparent adhesive film enables substantially all of the microscope light to be transferred to the mold, therein facilitating the identification of the quantity and quality of mold on the slide in the window. The laboratory technician records the data and returns the slide to the target zone, and then the next sample is analyzed. After all three samples have been analyzed and the slides are repositioned onto the slide mount adhesive, the leafs are refolded for storage of the kit. If, at a later time, further microscopic analysis is required, the kit can be retrieved.

What I claim is:

1. A mold test kit comprised of:
a) a foldable sheet of paperboard having leafs that can be folded into a multi-ply folder/mailer;
b) at least one paperboard slide perforatedly attached to the paperboard, said slide having a window with a pane that is a relatively tacky adhesive, supported on a clear film;
c) a removable cover slip that covers the window;
d) a reversibly attachable slide mounting zone with a filmic release layer for sending slides having sampled mold to a laboratory for analysis; and
e) printed instructions for use.

2. A mold test kit according to claim 1 further comprising: a low tack adhesive, wherein said low tack adhesive is deposited on the periphery of the at least one paperboard slide and augments the relatively tacky adhesive to adhere the cover slip to the paperboard slide.

3. The mold test kit as claimed in claim 1, wherein said relatively tacky adhesive supported on a clear film is a transparent pressure sensitive adhesive tape having a sufficient width and length to span the paperboard window, thereby providing the pane with the relatively tacky adhesive.

4. The mold test kit as claimed in claim 3, wherein said reversibly attachable slide mounting zone is comprised of: a pressure sensitive adhesive target tape adhered to the foldable sheet of paperboard; a slide mounting adhesive that is a longitudinal coating of pressure sensitive adhesive that is adjacent to an edge of the pressure sensitive adhesive target tape; and a release liner that protectively covers the slide mounting adhesive.

5. A method for using a mailable mold test kit, said method comprising the steps of:
opening the kit and recording on the kit the areas to be sampled for mold, wherein said mailable mold test kit is comprised of paperboard having leafs;
tearing off a slide along the perforations and peeling back/off the cover slide, therein exposing the window of tacky adhesive;
positioning the window over the area being sampled and pressing the window against the sample area, thereby pushing the tacky adhesive against the mold, enabling the adhesive to collect a sample of the mold;
mounting the slide on a slide mounting zone having a release layer, by exposing a portion of slide mounting adhesive that is coated along an edge of the slide mounting zone and protected with a release liner, by peeling back the release liner, and pressing an edge of the slide down, so that the slide is in contact with the slide mounting adhesive;
recording additional sampling data as needed;
repeating the sampling until all the samples are collected; and
folding the leafs into a mailer and mailing the samples to a designated lab for analysis.

6. A method for using a mailable mold test kit according to claim 5, said method further comprising the steps of:
receiving the sample at the lab;
removing the slide from the mounting zone, and microscopically examining the mold;
analyzing all samples for both mold type and quantity;
remounting the paperboard slide on the slide mounting zone with the mold against the paperboard and storing for further examination if required;
providing the interested parties with the results of the test.
